(19) United States
(12) Patent Application Publication
(10) Pub. No.: US 2009/0275963 A1
(43) Pub. Date: Nov. 5, 2009

(54) High-Strength Suture With Absorbable Components

(76) Inventors: Thomas C. May, Wrenthan, MA (US); Joseph Hotter, Middletown, CT (US); Jon Reinprecht, Watertown, CT (US); Richard Hammond, Northford, CT (US)

Correspondence Address: Tyco Healthcare Group LP, 60 MIDDLETOWN AVENUE, NORTH HAVEN, CT, 06473, US

(21) Appl. No.: 12/431,853

(22) Filed: Apr. 29, 2009

Related U.S. Application Data
(60) Provisional application No. 61/049,548, filed on May 1, 2008.

(51) Int. Cl. A61B 17/04; A61L 17/06

(52) U.S. Cl. 606/151; 606/228

(57) ABSTRACT

First and second yarns are interconnected to form surgical devices. The first yarns include a plurality of filaments including one or more filaments made from a high strength material and the second yarns include a plurality of filaments including one or more filaments made from an absorbable material.

### CROSS-REFERENCE TO RELATED APPLICATION

The present application claims the benefit of, and priority to, U.S. Provisional Patent Application Ser. No. 61/049,548 filed on May 1, 2008, the entire disclosure of which is incorporated by reference herein.

### BACKGROUND

1. Technical Field

The present disclosure relates to yarns that contain filaments made from high strength materials and/or absorbable materials, and braided multifilaments suitably adapted for use as surgical devices made from such yarns.

2. Background of Related Art

Braided multifilaments often offer a combination of enhanced pliability, knot security, and tensile strength when compared to their monofilament counterparts. The enhanced pliability of a braided multifilament is a direct consequence of the lower resistance to bending of a bundle of very fine filaments relative to one large diameter monofilament. However, a tradeoff between braid strength and pliability exists in the design of conventional braided multifilaments.

Braided multifilaments intended for the repair of body tissues should meet certain requirements: they should be substantially non-toxic, capable of being readily sterilized, possess good tensile strength and pliability, and have acceptable knot-tying and knot-holding characteristics. If the braided multifilaments are of the biodegradable variety, the degradation of the braided multifilaments should be predictable and closely controlled. Moreover, colored multifilaments may aid a surgeon during a surgical procedure by providing greater visibility of the device.

### SUMMARY

The present disclosure describes first and second yarns that are interconnected to form surgical devices. The yarns may be interconnected in a braided construction. In embodiments, the yarns are braided, knitted, or woven into a suture, mesh, sternal closure device, cable, tape or tether.

The first yarns include a plurality of filaments including one or more filaments made from a high strength material and the second yarns include a plurality of filaments including one or more filaments made from an absorbable material. In embodiments, the yarns of the present disclosure include homogenous yarns that include all high strength filaments or all absorbable filaments. The present disclosure also describes heterogeneous yarns that include a plurality of filaments made from a high strength material and one or more filaments made from an absorbable material, and in other embodiments, heterogeneous yarns that include a plurality of filaments made from an absorbable material and one or more filaments made from a high strength material. The surgical devices may include combinations of homogenous and heterogeneous yarns.

The absorbable filaments of the yarns may include a color element for identifying the surgical device. The color element may be a single uniform color, a gradation of color, multiple colors, a design pattern, or combinations thereof along a portion of the filaments of the surgical device. In embodiments, the color element is substantially the same among the filaments, in other embodiments the color element varies between filaments of the same or different yarns.

### BRIEF DESCRIPTION OF THE DRAWINGS

The accompanying drawings, which are incorporated in and constitute a part of the specification, illustrate embodiments of the disclosure and, together with a general description of the disclosure given above, and the detailed description of the embodiments given below, serve to explain the principles of the disclosure.

FIGS. 1A and 1B show illustrative embodiments of yarns in accordance with the present disclosure;

FIGS. 2A, 2B and 2C show illustrative embodiments of braids in accordance with the present disclosure; and

FIGS. 3A and 3B show illustrative embodiments of filaments including a color element for use in a surgical device as described herein.

### DETAILED DESCRIPTION OF THE EMBODIMENTS

Filaments made from high strength materials and absorbable materials are used in accordance with the present disclosure to prepare yarns that can be incorporated into a braided, knitted, woven, or other suitable structure to provide a surgical device.

A plurality of filaments is used to form a yarn. A plurality of yarns is used to form a braid, knit or weave.

A “heterogeneous yarn” is a configuration containing at least two dissimilar filaments mechanically bundled together to form a yarn. The filaments are continuous and discrete, so therefore each filament extends substantially along the entire length of the yarn and maintains its individual integrity during yarn preparation, processing, and use.

Unlike a heterogeneous yarn, a “homogeneous” yarn is a configuration containing substantially similar filaments. The filaments are also continuous and discrete. Therefore each filament extends substantially along the entire length of the yarn and maintains its individual integrity during yarn preparation, processing, and use.

A “heterogeneous braid” is a configuration containing at least two dissimilar yarns. The two types of yarns are intertwined in a braided construction. The yarns are continuous and discrete, so therefore each yarn extends substantially along the entire length of the braid and maintains its individual integrity during braid preparation, processing, and use.

A “homogeneous braid” then, is a configuration containing substantially similar yarns. The yarns are intertwined in a braided construction. The yarns are continuous and discrete. Therefore each yarn extends substantially along the entire length of the braid and maintains its individual integrity during braid preparation, processing, and use.

In the broadest sense, this disclosure contemplates yarns that include at least one filament made from a high strength material and yarns that include at least one filament made from an absorbable material, articles made therefrom, and their use in surgery. Methods for forming filaments from high strength materials as well as filaments from absorbable materials are within the purview of those skilled in the art. The yarns can be a homogeneous yarn made entirely of either a high strength material or an absorbable material. In other embodiments, the yarns are heterogeneous. The yarns may be made from at least one high strength filament or absorbable filament in combination with a plurality of filaments made from at least one other fiber forming material. For example, the yarns may include a combination of high strength and absorbable materials.

High strength materials include extended chain fibers having a molecular weight of at least about 500,000 g/mole. In embodiments, the high strength material has a molecular weight between about 1,000,000 g/mole and about 5,000,000 g/mol, in embodiments between about 2,000,000 g/mole and about 4,000,000 g/mole. Examples of high strength polymers include, for example, ethylene vinyl acetate, poly(meth)acrylic acid, polyester, polyamides, polyethylene, polypropylene, polystyrene, polyvinyl chloride, polyvinylphenol, polyacrylonitrile, and copolymers and mixtures thereof. A particularly suitable non-biodegradable high strength fiber is ultra high molecular weight polyethylene, available under the tradename SPECTRA® (Honeywell, Inc., Morristown, N.J.). Other ultra high molecular weight polyethylene sutures are disclosed, for example, in U.S. Pat. No. 5,318,575, the entire contents of which are incorporated herein by reference.

Absorbable materials are absorbed by biological tissues and disappear in vivo at the end of a given period, which can vary for example from one day to several months, depending on the chemical nature of the material. Absorbable materials include both natural and synthetic biodegradable polymers.

Representative natural biodegradable polymers include polysaccharides such as alginate, dextran, cellulose, collagen, and chemical derivatives thereof (substitutions, additions of chemical groups, for example, alkyl, alkylene, hydroxylations, oxidations, and other modifications routinely made by those skilled in the art), and proteins such as albumin, zein and copolymers and blends thereof, alone or in combination with synthetic polymers.

Synthetically modified natural polymers include cellulose derivatives such as alkyl celluloses, hydroxyalkyl celluloses, cellulose ethers, cellulose esters, nitrocelluloses, and chitosan. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, and cellulose sulfate sodium salt. These are collectively referred to herein as “celluloses.” Representative synthetic degradable polymers include polyhydroxy acids prepared from lactone monomers such as glycolide, lactide, trimethylene carbonate, p-dioxanone, ε-caprolactone, and combinations thereof. Polymers formed therefrom include, for example, polylactides, polyglycolides, and copolymers thereof; poly(hydroxybutyric acid); poly(hydroxyvaleric acid); poly(lactide-co-(ε-caprolactone-)); poly(glycolide-co-(ε-caprolactone)); polycarbonates; poly(pseudo amino acids); poly(amino acids); poly(hydroxyalkanoate)s; polyanhydrides; polyortho esters; and blends and copolymers thereof.

Rapidly bioerodible polymers such as poly(lactide-co-glycolide)s, polyanhydrides, and polyorthoesters, which have carboxylic groups exposed on the external surface as the smooth surface of the polymer erodes, may also be used.

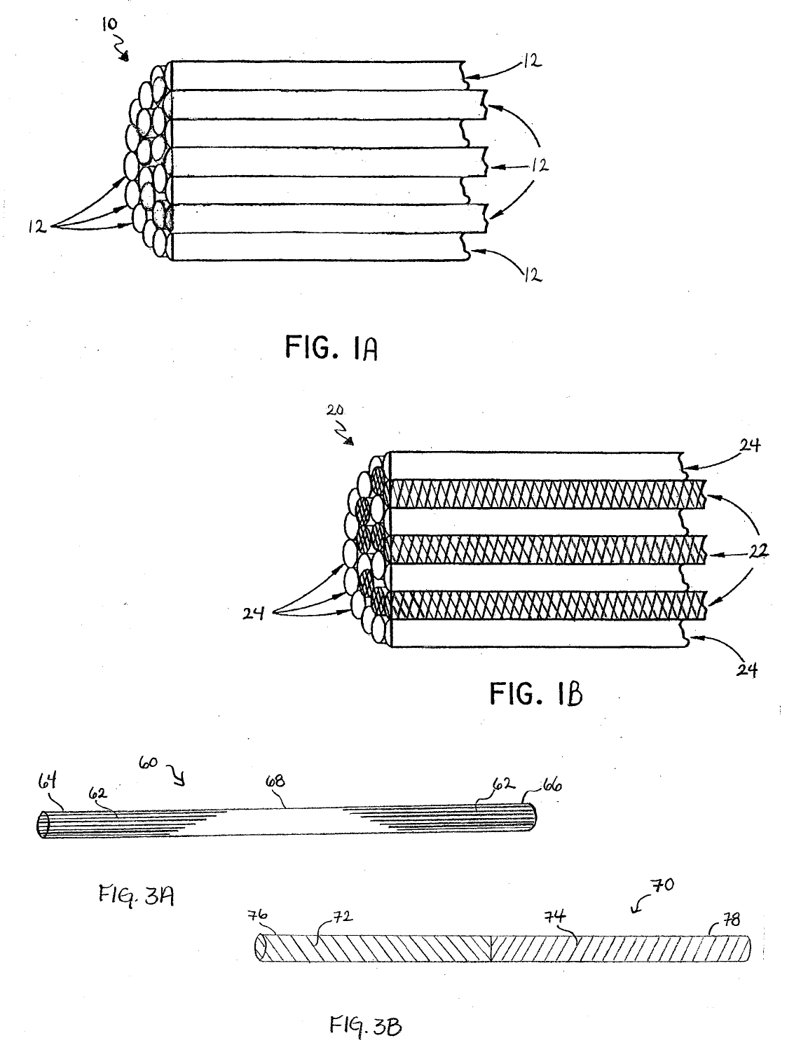

Turning now to FIGS. 1A and 1B, a plurality of filaments are commingled to form yarns. The filaments may be systematically or randomly arranged within a yarn, such as by twisting, plaiting, braiding, or laying the filaments substantially parallel to form the yarn. FIG. 1A illustrates a homogeneous yarn 10 including a plurality of substantially similar filaments 12. In embodiments, homogeneous yarn 10 includes a plurality of high strength filaments, and in other embodiments, homogeneous yarn 10 includes a plurality of absorbable filaments.

A heterogeneous yarn 20, on the other hand, contains a plurality of two dissimilar filaments 22, 24 as shown in FIG. 1B. In embodiments, first filaments 22 are made from a high strength material and second filaments 24 are made from an absorbable material. In other embodiments, first filaments 22 may be made from a high strength material or from an absorbable material and second filaments 24 may be formed from other fiber forming materials, such as non-degradable polymers like shape memory polymer or alloys.

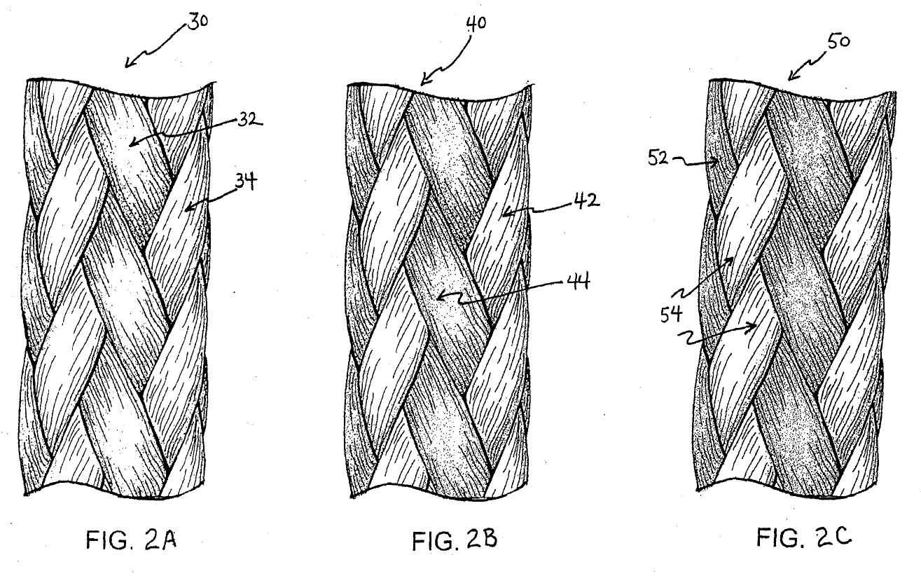

Referring now to FIGS. 2A-2C, braids are formed from yarns. As shown in FIG. 2A, a braid 30 contains two similar heterogeneous yarns 32, 34. Each heterogeneous yarn contains a plurality of two dissimilar filaments. In embodiments, a first filament is a high strength material and a second filament is made from an absorbable material. The yarns 32, 34 are intertwined to form a substantially homogeneous braid 30.

FIG. 2B illustrates a heterogeneous braid 40 containing two dissimilar yarns 42, 44. In embodiments, a first yarn 42 contains a plurality of filaments made from a high strength material and a second yarn 44 contains a plurality of filaments made from an absorbable material. The homogeneous first and second yarns 42, 44 are intertwined to form a heterogeneous braid 40.

In another embodiment shown in FIG. 2C, a heterogeneous braid 50 contains a heterogeneous yarn 52 and a homogeneous yarn 54. As described above, a heterogeneous yarn contains a plurality of two dissimilar filaments. In embodiments, a first filament is made from a high strength material and a second filament is made from an absorbable material. The homogeneous yarn contains a plurality of filaments made from any material capable of being spun into a filament. The heterogeneous yarn 52 and the homogeneous yarn 54 are intertwined to form a heterogeneous braid 50.

A braid and/or yarn can be prepared using conventional braiding, weaving, or other technology and equipment commonly used in the textile industry and in the medical industry for preparing multifilament sutures. Suitable braid constructions include, for example tubular, hollow, and spiroid braids and are disclosed, for example, in U.S. Pat. Nos. 3,187,752; 3,565,077; 4,014,973; 4,043,344; 4,047,533; 5,019,093; and 5,059,213, the disclosures of which are incorporated herein by reference. Illustrative flat braided structures (suitable, e.g., for tendon repair) which can be formed using the presently described yarns include those described in U.S. Pat. Nos. 4,792,336 and 5,318,575. Suitable mesh structures are shown and described, for example, in U.S. Pat. No. 5,292,328.

If desired, the surface of a filament, yarn, or braid can be coated with a bioabsorbable or nonabsorbable coating to further improve the performance of the braid. For example, a braid can be immersed in a solution of a desired coating polymer in an organic solvent, and then dried to remove the solvent.

A braid is sterilized so it can be used for a host of medical applications, especially for use as a surgical suture, cable, tether, tape and sternal closure device, which may be attached to a needle, suture anchor, or bone anchor.

Once sterilized, a braided multifilament surgical device, as described herein, may be used to repair wounds located between two or more soft tissues, two or more hard tissues, or at least one soft tissue and at least one hard tissue. The braided multifilament surgical device is passed through, wrapped around or secured to tissue and then the tissue is approximated by manipulating the braided multifilament surgical device, such as, for example, by tying a knot, cinching the device, applying a buckle, or the like.

In embodiments, a braid is made of heterogeneous yarns to form a surgical suture. The heterogeneous yarns contain filaments made from high strength materials and filaments made from absorbable materials. In embodiments, the heterogeneous yarns contain one or more high strength materials and one or more absorbable materials. In other embodiments, the braid may contain two sets of yarns, each containing different high strength and absorbable filaments. The high strength filaments may comprise from about 5% to about 95% of the cross-sectional area of the heterogeneous yarns, in embodiments from about 25% to about 75%, and in other embodiments from about 40% to about 60% of the heterogeneous yarns. The braid may be composed of yarns having the same or different proportion of high strength filaments to absorbable filaments.

In an embodiment, the heterogeneous yarns include filaments made from ultra high molecular weight polyethylene and filaments made from copolymers of glycolide and lactide, the ultra high molecular weight polyethylene filaments comprising about 10% to about 90% of the braid, in embodiments about 25% to about 75% of the braid, and in other embodiments about 30% to about 55% of the braid.

Sutures made in accordance with the foregoing description will exhibit superior strength and handling properties, as well as reduced long term implantable mass. High strength fibers, particularly ultra high molecular weight polyethylene, have a high tensile strength but an inherently low coefficient of friction thereby exhibiting poor knot security. Improved knot security is obtained by braiding absorbable filaments, which have a higher surface friction than high strength materials, into the device along with the high strength filaments. Additionally, absorbable filaments degrade after implantation, whereas high strength filaments are generally non-degradable. Thus, the braided suture would have less mass remaining long term after implantation.

In embodiments, the braided multifilaments include a color element to enhance visibility of the device. Ultra high molecular weight polyethylene filaments are substantially translucent or colorless and are currently only available without coloration. Absorbable filaments, on the other hand, can be colored via a number of conventional ways to produce a variety of different colors and/or color patterns. For example, a color element may be coated, sprayed, glued, dyed, stained, or otherwise affixed onto and/or into the absorbable material. By combining the translucent high strength filaments with colored absorbable filaments, color variation is achieved resulting visually identifiable and distinguishable sutures.

The color element may appear in various forms to provide visual identification and/or differentiation of the suture. Absorbable filaments are available in a variety of colors to visually distinguish sutures or to allow yarns to be woven into a wide variety of distinguishable patterns. In embodiments, all or a portion of the absorbable filaments in a yarn and/or braid may comprise a single uniform color. In other embodiments, all or a portion of the absorbable filaments in a yarn and/or braid may have a gradation of color, multiple colors, a design pattern, or combinations thereof. For example, as illustrated in FIG. 3A, the intensity of the color element 62 may decrease from the end portions 64, 66 of a filament 60 toward the middle portion 68 for visually identifying the location of an end portion. In an embodiment, the filament may include sections having different lengths of color where the spacing between the adjacent sections increase from one end toward the other end of the filament. In another exemplary embodiment shown in FIG. 3B, filament 70 may include a first color element 72 disposed on a first portion 76 of the filament 70 and a second color element 74 disposed on a second portion 78 of the filament 70. In yet other embodiments, the color element may also be in the form of a pattern, such as shapes or arrangements that afford a different identification effect. For example, the color may extend along the length of the absorbable filament in a spiral pattern or as a plurality of stripes. It is envisioned that that the individual filaments within a yarn or braid may have different color elements and that the yarns within a braid may have different numbers of filaments containing color elements to form a visually distinct braid. It will be appreciated that other embodiments of color elements of an absorbable filament are also within the scope of the present disclosure.

In embodiments, the suture may also be a vehicle for delivery of pharmaceutical agents. A pharmaceutical agent as used herein is used in the broadest sense and includes any substance or mixture of substances that have clinical use. Consequently, pharmaceutical agents may or may not have pharmacological activity per se, e.g., a dye or fragrance. Alternatively a pharmaceutical agent could be any bioactive agent which provides a therapeutic or prophylactic effect, a compound that affects or participates in tissue growth, cell growth, cell differentiation, an anti-adhesive compound, a compound that may be able to invoke a biological action such as an immune response, or could play any other role in one or more biological processes. A variety of pharmaceutical agents may be incorporated into a coating and/or into the bulk polymer structure.

Examples of classes of pharmaceutical agents which may be utilized in accordance with the present disclosure include anti-adhesives, antimicrobials, analgesics, antipyretics, anesthetics, antiepileptics, antihistamines, anti-inflammatories, cardiovascular drugs, diagnostic agents, sympathomimetics, cholinomimetics, antimuscarinics, antispasmodics, hormones, growth factors, muscle relaxants, adrenergic neuron blockers, antineoplastics, immunogenic agents, immunosuppressants, gastrointestinal drugs, diuretics, steroids, lipids, lipopolysaccharides, polysaccharides, platelet activating drugs, clotting factors, and enzymes. It is also intended that combinations of agents may be used.

Other pharmaceutical agents which may be included as a bioactive agent in the coating composition applied in accordance with the present disclosure include: local anesthetics; non-steroidal antifertility agents; parasympathomimetic agents; psychotherapeutic agents; tranquilizers; decongestants; sedative hypnotics; steroids; sulfonamides; sympathomimetic agents; vaccines; vitamins; antimalarials; anti-migraine agents; anti-parkinson agents such as L-dopa; anti-spasmodics; anticholinergic agents (e.g. oxybutynin); antitussives; bronchodilators; cardiovascular agents such as coronary vasodilators and nitroglycerin; alkaloids; analgesics; narcotics such as codeine, dihydrocodeinone, meperidine, morphine and the like; non-narcotics such as salicylates, aspirin, acetaminophen, d-propoxyphene and the like; opioid receptor antagonists, such as naltrexone and naloxone; anti-cancer agents; anti-convulsants; anti-emetics; antihistamines; anti-inflammatory agents such as hormonal agents, hydrocortisone, prednisolone, prednisone, non-hormonal agents, allopurinol, indomethacin, phenylbutazone and the like; prostaglandins and cytotoxic drugs; chemotherapeutics, estrogens; antibacterials; antibiotics; anti-fungals; anti-virals; anticoagulants; anticonvulsants; antidepressants; and immunological agents.

Other examples of suitable pharmaceutical agents which may be included in the coating composition include viruses and cells, peptides, polypeptides and proteins, analogs, muteins, and active fragments thereof, such as immunoglobulins, antibodies, cytokines (e.g. lymphokines, monokines, chemokines), blood clotting factors, hemopoietic factors, interleukins (IL-2, IL-3, IL-4, IL-6), interferons (β-IFN, (α-IFN and γ-IFN), erythropoietin, nucleases, tumor necrosis factor, colony stimulating factors (e.g., GCSF, GM-CSF, MCSF), insulin, anti-tumor agents and tumor suppressors, blood proteins, fibrin, thrombin, fibrinogen, synthetic thrombin, synthetic fibrin, synthetic fibrinogen, gonadotropins (e.g., FSH, LH, CG, etc.), hormones and hormone analogs (e.g., growth hormone), vaccines (e.g., tumoral, bacterial and viral antigens), somatostatin, antigens, blood coagulation factors, growth factors (e.g., nerve growth factor, insulin-like growth factor), bone morphogenic proteins, TGF-B, protein inhibitors, protein antagonists, and protein agonists, nucleic acids, such as antisense molecules, DNA, RNA, and RNAi, oligonucleotides, polynucleotides, and ribozymes.

Various modifications and variations of the yarns, braids and devices and uses thereof will be apparent to those skilled in the art from the foregoing detailed description. Such modifications and variations are intended to come within the scope of the following claims.

What is claimed is:

1. A surgical device comprising:
first yarns comprising a plurality of filaments including at least one filament made from a high strength material; and
second yarns comprising a plurality of filaments including at least one filament made from an absorbable material,
wherein the first yarns are interconnected with the second yarns.

2. The surgical device according to claim 1, wherein the high strength material has a molecular weight between about 500,000 g/mole and 5,000,000 g/mol.

3. The surgical device according to claim 1, wherein the high strength material is selected from the group consisting of ethylene vinyl acetate, poly(meth)acrylic acid, polyamides, polyethylene, polypropylene, polystyrene, polyvinyl chloride, polyvinylphenol, and copolymers and mixtures thereof.

4. The surgical device according to claim 1, wherein the high strength material is ultra high molecular weight polyethylene.

5. The surgical device according to claim 1, wherein the absorbable material is selected from the group consisting of polysaccharides, proteins, cellulose derivatives, polyhydroxy acids, and blends, copolymers and mixtures thereof.

6. The surgical device according to claim 1, wherein the absorbable material is selected from the group consisting of glycolide, lactide, trimethylene carbonate, p-dioxanone, ε-caprolactone, co-polymers and combinations thereof.

7. The surgical device according to claim 1, wherein all the filaments of the first yarns are made from a high strength material.

8. The surgical device according to claim 1, wherein all the filaments of the second yarns are made from an absorbable material.

9. The surgical device according to claim 7, wherein all the filaments of the second yarns are made from an absorbable material.

10. The surgical device according to claim 1, wherein the first yarns are heterogeneous yarns comprising a plurality of filaments made from a high strength material and at least one filament made from an absorbable material.

11. The surgical device according to claim 1, wherein the second yarns are heterogeneous yarns comprising filaments made from an absorbable material and filaments made from a high strength material.

12. The surgical device according to claim 10, wherein the second yarns are heterogeneous yarns comprising a plurality of filaments made from an absorbable material and at least one filament made from a high strength material.

13. The surgical device according to claim 9, wherein the absorbable filaments of the second yarns include a color element for identifying the device.

14. The surgical device according to claim 13, wherein the color element is selected from a single uniform color, a gradation of color, multiple colors, a design pattern, or combinations thereof along a portion of the absorbable filaments of the surgical device.

15. The surgical device according to claim 10, wherein the absorbable filaments of the first yarns include a color element for identifying the surgical device.

16. The surgical device according to claim 12, wherein the absorbable filaments of the first yarns include a first color element and the absorbable filaments of the second yarns include a second color element, wherein the first color element is different than the second color element.

17. The surgical device according to claim 1, wherein the first and second yarns are interconnected in a braided construction.

18. The surgical device according to claim 1, wherein the first and second yarns are configured and dimensioned to form a device selected from the group consisting of a suture, mesh, sternal closure device, cable, tape and tether.
